(19) United States
(12) Patent Application Publication
(10) Pub. No.: US 2017/0260571 A1
(43) Pub. Date: Sep. 14, 2017

(54) METHODS AND MATERIALS FOR TREATING ENDOMETRIAL CANCER

(71) Applicant: Mayo Foundation for Medical Education and Research, Rochester, MN (US)

(72) Inventors: Andrea Mariani, Rochester, MN (US); Nicholas Chia, Rochester, MN (US); Marina R. Walther-Antonio, Rochester, MN (US)

(73) Assignee: Mayo Foundation for Medical Education and Research, Rochester, MN (US)

(21) Appl. No.: 15/451,845

(22) Filed: Mar. 7, 2017

Related U.S. Application Data
(60) Provisional application No. 62/305,075, filed on Mar. 8, 2016.

(51) Int. Cl. C12Q 1/68; A61K 45/06; A61K 31/567

(52) U.S. Cl. CPC C12Q 1/689; A61K 31/567; A61K 45/06; C12Q 2600/158

(57) ABSTRACT

This document provides methods and materials for detecting and/or treating endometrial hyperplasia and/or endometrial cancer. For example, this document provides methods and materials for identifying a female mammal as having endometrial hyperplasia and administering a therapy (e.g., hormone therapy) to the female mammal. For example, this document provides methods and materials for identifying a female mammal as having endometrial cancer and surgically removing at least the uterus of the female mammal.

### CLAIM OF PRIORITY

This application claims the benefit of U.S. Patent Application Ser. No. 62/305,075, filed on Mar. 8, 2016. The entire contents of which are hereby incorporated by reference.

### TECHNICAL FIELD

This document relates to methods and materials involved in detecting and/or treating endometrial hyperplasia and/or endometrial cancer. For example, this document provides methods and materials for identifying a female mammal as having endometrial hyperplasia and administering a therapy (e.g., hormone therapy) to the female mammal. This document also provides methods and materials for identifying a female mammal as having endometrial cancer and surgically removing at least the uterus of the female mammal.

### BACKGROUND

Endometrial cancer is the most common gynecologic cancer currently afflicting an estimated 600,000 women in the U.S. alone, with 49,000 new cases identified and 8,000 deaths in 2013. Genetic risk factors, such as hereditary non-polyposis colorectal cancer, explain less than 5% of all endometrial cancer cases (Lancaster et al., Gynecol. Oncol., 107(2):159-162 (2007) and Kwon et al., J. Clin. Oncol., 29(16):2247-2252 (2011)). Multiple non-genetic risk factors for endometrial cancer exist. These risk factors can be broadly categorized into those linked to hormonal changes, such as obesity and hormone therapy, and those linked to chronic inflammation, such as diabetes and aging (Smith et al., Cancer J. Clinicians, 51(1):38-75 (2001)).

### SUMMARY

This document provides methods and materials for detecting and/or treating endometrial hyperplasia and/or endometrial cancer. For example, this document provides methods and materials for identifying a female mammal as having endometrial hyperplasia and administering a therapy (e.g., hormone therapy) to the female mammal. This document also provides methods and materials for identifying a female mammal as having endometrial cancer and surgically removing at least the uterus of the female mammal. In some cases, the materials and methods described herein provide a minimally invasive early screening tool for the detection and/or treatment of endometrial hyperplasia and/or endometrial cancer.

As described herein, female mammals can be identified as having endometrial hyperplasia based at least in part on the presence of an Atopobium species (e.g., A. vaginae) and the presence of a Porphyromonas species, within the upper reproductive tract, and/or female mammals can be identified as having endometrial cancer based at least in part on the presence of an Atopobium species (e.g., A. vaginae), and optionally the presence of a Porphyromonas species, in the lower reproductive tract reproductive tract. In some cases, female mammals having endometrial hyperplasia and/or endometrial cancer can have a high vaginal pH (e.g., greater than 4.5, greater than 5.0, or greater than 5.5).

Once identified as having endometrial hyperplasia and/or endometrial cancer as described herein, a therapy can be administered to a female mammal. For example, a female mammal identified as having endometrial hyperplasia can be treated by administering a progesterone hormone therapy (e.g., cyclic or continuous progestin therapy). For example, a female mammal identified as having endometrial cancer as described herein can be treated by surgical removal of at least the uterus of the female mammal (e.g., a hysterectomy such as a vaginal hysterectomy, an abdominal hysterectomy, a total laparoscopic hysterectomy, a total hysterectomy with lateral or bilateral salpingo-oophorectomy, or a radical hysterectomy). In some cases, a female mammal identified as having endometrial cancer as described herein can be treated using radiation therapy (e.g., high-energy x-ray therapy), chemotherapy (e.g., paclitaxel, carboplatin, doxorubicin, and/or cisplatin therapy), or hormone therapy (e.g., tamoxifen, goserelin, leuprolide, exemestane, anastrozole, and/or letrozole therapy) in combination with or in place of surgery.

In general, one aspect of this document features a method for treating a female mammal having endometrial cancer. The method includes, or consists essentially of, identifying the mammal as having an Atopobium species and/or a Peptoniphilus species is within the lower reproductive tract of the mammal, and removing the uterus of the mammal. The mammal can be a human. The Atopobium species can be A. vaginae. The Peptoniphilus species can be P. harei or P. coxii. The method also can include identifying the mammal as having a Porphyromonas species within the lower reproductive tract of the mammal. The Porphyromonas species can be P. somerae. The Porphyromonas species can be a species having 16S rRNA that is greater than 98 percent (e.g., greater than 98.5 percent) identical to a 16S rRNA sequence of P. somerae.

In another aspect, this document features a method for identifying a female mammal as having endometrial cancer. The method includes, or consists essentially of, detecting the presence of an Atopobium species and/or a Peptoniphilus species within the lower reproductive tract of the mammal, and classifying the mammal as having endometrial cancer. The mammal can be a human. The Atopobium species can be A. vaginae. The Peptoniphilus species can be P. harei or P. coxii. The method can include detecting the presence of a Porphyromonas species within the lower reproductive tract of the mammal. The Porphyromonas species can be P. somerae. The Porphyromonas species can be a species having 16S rRNA that is greater than 98 percent (e.g., greater than 98.5 percent) identical to a 16S rRNA sequence of P. somerae.

In another aspect, this document features a method for treating a female mammal having endometrial hyperplasia. The method includes, or consists essentially of, identifying the mammal as having an Atopobium species, a Porphyromonas species, and or a Peptoniphilus species within the upper reproductive tract of the mammal, but not within the lower reproductive tract of the mammal, and administering progestin therapy to the mammal. The mammal can be a human. The Atopobium species can be A. vaginae. The Porphyromonas species can be P. somerae. The Porphyromonas species can be a species having 16S rRNA that is greater than 98 percent (e.g., greater than 98.5 percent) identical to a 16S rRNA sequence of P. somerae. The Peptoniphilus species can be P. harei or P. coxii.

In another aspect, this document features a method for identifying a female mammal as having endometrial hyperplasia. The method includes, or consists essentially of, detecting the presence of an Atopobium species, a Porphyromonas species, and/or a Peptoniphilus species within the upper reproductive tract of the mammal, but not within the lower reproductive tract of the mammal, and classifying the mammal as having endometrial hyperplasia. The mammal can be a human. The Atopobium species can be A. vaginae. The Porphyromonas species can be P. somerae. The Porphyromonas species can be a species having 16S rRNA that is greater than 98 percent (e.g., greater than 98.5 percent) identical to a 16S rRNA sequence of P. somerae. The Peptoniphilus species can be P. harei or P. coxii.

In another aspect, this document features a method for treating a female mammal having endometrial cancer. The method includes, or consists essentially of, identifying the mammal as having an Atopobium species and/or a Peptoniphilus species within the lower reproductive tract of the mammal, and administering an antibiotic to the mammal to reduce the number of Atopobium species bacteria present within the lower reproductive tract of the mammal. The mammal can be a human. The Atopobium species can be A. vaginae. The Peptoniphilus species can be P. harei or P. coxii. The method also can include identifying the mammal as having a Porphyromonas species within the lower reproductive tract of the mammal. The Porphyromonas species can be P. somerae. The Porphyromonas species can be a species having 16S rRNA that is greater than 98 percent (e.g., greater than 98.5 percent) identical to a 16S rRNA sequence of P. somerae. The antibiotic can be benzylpenicillin, tetracycline, amoxicillin, ampicillin, ticarcillin, piperacillin, cephalothin, cefuroxime, cefotaxime, cefoxitin, imipenem erythromycin, cefamandole, cephaloridine, oleandomycin, metronidazole, spiramycin, or clindamycin.

In another aspect, this document features a method for treating a female mammal as having endometrial hyperplasia. The method includes, or consists essentially of, identifying the mammal as having an Atopobium species, a Porphyromonas species, and/or a Peptoniphilus species within the upper reproductive tract of the mammal, but not within the lower reproductive tract of the mammal, and administering an antibiotic to the mammal to reduce the number of Atopobium species bacteria, the number of Porphyromonas species bacteria, and/or the number of Peptoniphilus species bacteria present within the lower reproductive tract of the mammal. The mammal can be a human. The Atopobium species can be A. vaginae. The Porphyromonas species can be P. somerae. The Porphyromonas species can be a species having 16S rRNA that is greater than 98 percent (e.g., greater than 98.5 percent) identical to a 16S rRNA sequence of P. somerae. The Peptoniphilus species can be P. harei or P. coxii. The antibiotic can be benzylpenicillin, tetracycline, amoxicillin, ampicillin, ticarcillin, piperacillin, cephalothin, cefuroxime, cefotaxime, cefoxitin, imipenem erythromycin, cefamandole, cephaloridine, oleandomycin, metronidazole, spiramycin, or clindamycin. The method can include administering progestin therapy to the mammal.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 shows endometrial microbiomes across cohorts. Only taxa present at a minimum of 5% relative frequency in at least one subject are shown for graphical clarity. Taxa color scheme reflects abundance relative to each patient (darker coloration represents higher abundance). Abbreviations: Meno/Menometrorrhagia—Menorrhagia/Menometrorrhagia; Dysme—Dysmenorrhagia/Pelvic Pain; W/ Aty—With Atypia; Muci—Mucinous; Squa—Squamous; Carcino—Carcinosarcoma; Hyper—Hyperplasia.

FIG. 2 shows vaginal/cervical (lower tract) microbiomes across cohorts. Only taxa present at a minimum of 5% relative frequency in at least one subject are shown for graphical clarity. Taxa color scheme reflects abundance relative to each patient (darker coloration represents higher abundance). Abbreviations: Dysme—Dysmenorrhagia/Pelvic Pain; W/ Aty—With Atypia; Muci—Mucinous; Squa—Squamous; Hyper—Hyperplasia.

FIG. 3 shows fallopian tube microbiomes across cohorts. Only taxa present at a minimum of 5% relative frequency in at least one subject are shown for graphical clarity. Taxa color scheme reflects abundance relative to each patient (darker coloration represents higher abundance). Abbreviations: Meno/Menometrorrhagia—Menorrhagia/Menometrorrhagia; Dysme—Dysmenorrhagia/Pelvic Pain; W/o Aty—Without Atypia; W/ Aty—With Atypia; Muci—Mucinous; Squa—Squamous; Hyper—Hyperplasia.

FIG. 4 shows ovarian microbiomes across cohorts. Only taxa present at a minimum of 5% relative frequency in at least one subject are shown for graphical clarity. Taxa color scheme reflects abundance relative to each patient (darker coloration represents higher abundance). Abbreviations: Dysme—Dysmenorrhagia/Pelvic Pain; W/Aty —With Atypia; Squa—Squamous; Hyper—Hyperplasia.

FIGS. 5A and 5B shows α-diversity comparisons between different disease states in the endometrial microbiome. Error bars represent the standard errors. A. Observed OTU number. B. Shannon index.

FIGS. 6A and 6B shows α-diversity for the lower tract. Error bars represent the standard errors. A. Observed OTU number. B. Shannon index.

FIG. 7 shows an ordination plot based on weighted UniFrac distance depicting the relationship between different disease states. Each point represents a sample and is colored by sample group.

FIG. 8 shows a maximum likelihood phylogenetic tree of the V3-V5 16S rDNA region of the recovered Porphyromonas sp. ^{a}Recovered from children with atopic dermatitis; ^{b}Recovered from buffaloes with postpartum endometritis; ^{c}Recovered from Holstein dairy cows with postpartum metritis. Produced with FASTTREE.

FIG. 9 shows a ROC curve for Atopobium vaginae and Porphyromonas sp. presence in the lower reproductive tract (vagina/cervix) and disease status (benign vs endometrial cancer).

FIGS. 10A, 10B, and 10C show example collections. Only taxa present at more than 5% relative frequency per sample are shown for graphical clarity. A. Patient B02. B. Patient H72. C. Patient EC19.

FIG. 11 shows a schematic representation of pathogenesis in the presence of a Porphyromonas sp. and Atopobium vaginae in the reproductive tract.

### DETAILED DESCRIPTION

This document provides methods and materials for detecting and/or treating endometrial hyperplasia and/or endometrial cancer. As described herein, several taxa were found to be significantly enriched in the reproductive tract of female mammals having endometrial hyperplasia and/or endometrial cancer including, for example, Firmicutes (Anaerostipes, ph2, Dialister, Peptoniphilus, 1-68, Ruminococcus, Anaerococcus, Peptococcus, and Anaerotruncus), Spirochaetes (Treponema), Actinobacteria (Atopobium), Bacteroidetes (Bacteroides and Porphyromonas), and Proteobacteria (Arthrospira). In some cases, female mammals can be identified as having endometrial hyperplasia based at least in part on the presence of an Atopobium species (e.g., A. vaginae), a Porphyromonas species, and/or a Peptoniphilus species (e.g., P. harei or P. coxii) within the upper reproductive tract. In some cases, female mammals can be identified as having endometrial cancer based at least in part on the presence of an Atopobium species (e.g., A. vaginae), a Porphyromonas species, and/or a Peptoniphilus species (e.g., P. harei or P. coxii) in the lower reproductive tract reproductive tract. In some cases, female mammals having endometrial hyperplasia and/or endometrial cancer can have a high vaginal pH (e.g., greater than 4.5, greater than 5.0, or greater than 5.5).

Any type of female mammal can be assessed and/or treated as described herein. For example, the methods and materials described herein can be used for detecting and/or treating endometrial hyperplasia and/or endometrial cancer in female humans and other primates such as monkeys. In some cases, the methods and materials described herein can be used for detecting and/or treating endometrial hyperplasia and/or endometrial cancer in female dogs, cats, horses, cows, pigs, sheep, rabbits, mice, and rats.

In some cases, a female mammal assessed and/or treated as described herein can be a female human having a history of postmenopausal uterine bleeding and obesity (e.g., a 45-74 years old female with BMI≧30).

A reproductive tract sample can be an upper or lower reproductive tract sample. For example, an upper reproductive tract sample can be a sample obtained from a uterine swab, scrape, or biopsy. In some cases, an upper reproductive tract sample can be an endometrial, ovarian, fallopian tube, or peritoneal wash sample. A lower reproductive tract sample can be a sample obtained from a vaginal and/or cervical swab and scrape. Alternative samples can include, without limitation, rectal and oral samples, and can be obtained using, for example, a swab. A reproductive tract sample can be obtained from a posterior or superior sampling position. In some cases, bacterial cells and/or DNA can be separated from mammalian cells and/or DNA in the reproductive tract sample. For example, bacterial DNA enrichment can be used to separate bacterial DNA from human DNA in reproductive tract samples.

As described herein, detecting the presence of an Atopobium species, a Porphyromonas species, and/or a Peptoniphilus species within an upper reproductive tract sample from a female mammal, but not detecting the presence of an Atopobium species, a Porphyromonas species, and/or a Peptoniphilus species within a lower reproductive tract sample from the same female mammal, can indicate that the female mammal has endometrial hyperplasia, while detecting the presence of an Atopobium species, a Porphyromonas species, and/or a Peptoniphilus species within a lower reproductive tract sample from a female mammal can indicate that the female mammal has endometrial cancer. The Atopobium species can be any appropriate Atopobium species (e.g., A. vaginae). The Porphyromonas species can be any appropriate Porphyromonas species (e.g., P. somerae). In some cases, the Porphyromonas species can be a species having 16S rRNA that is greater than 98 percent identical (e.g., greater than 98 percent identical, greater than 98.5 percent identical, greater than 99 percent identical, or greater than 99.5 percent identical) to a 16S rRNA sequence of P. somerae. See, e.g., WO 2015/148909. A P. somerae strain designated WAL 6690 is available from the ATCC® (ATCC® deposit number: BAA-1230™). See, also, Summanen et al., J. Clin. Microbiol 43(9):4455 (2005)). The Peptoniphilus species can be any appropriate Peptoniphilus species (e.g., P. harei or P. coxii). In some cases, the presence of an Atopobium species, a Porphyromonas species, and/or a Peptoniphilus species within an upper reproductive tract sample and the absence of an Atopobium species, a Porphyromonas species, and/or a Peptoniphilus species within a lower reproductive tract sample can indicate that the female mammal has endometrial hyperplasia. In some cases, the presence of an Atopobium species, a Porphyromonas species, and/or a Peptoniphilus species within a lower reproductive tract sample can indicate that the female mammal has endometrial cancer. In some cases, endometrial biopsies, dilatation and curettage procedures, transvaginal ultrasound examinations, or combinations thereof can be used to confirm that a female has endometrial hyperplasia or endometrial cancer.

Any appropriate method can be used to detect the presence of an Atopobium species (e.g., A. vaginae), a Porphyromonas species, and/or a Peptoniphilus species (e.g., P. harei or P. coxii) within a reproductive tract sample. For example, PCR-based assays designed to amplify nucleic acid of an Atopobium species can be used to detect the presence of an Atopobium species within a reproductive tract sample. For example, PCR-based assays designed to amplify nucleic acid of a Porphyromonas species can be used to detect the presence of a Porphyromonas species within a reproductive tract sample. For example, PCR-based assays designed to amplify nucleic acid of a Peptoniphilus species can be used to detect the presence of a Peptoniphilus species within a reproductive tract sample. In some cases, rRNA nucleic acid can be amplified and sequenced to detect the presence of an Atopobium species, a Porphyromonas species, and/or a Peptoniphilus species within a reproductive tract sample

In some cases, a PCR-based technique can be performed to amplify nucleic acid from an Atopobium species, a Porphyromonas species, and/or a Peptoniphilus species. Once amplified, the nucleic acid can be sequenced to confirm the presence of nucleic acid from an Atopobium species, a Porphyromonas species, and/or a Peptoniphilus species. In some cases, a PCR primer pair or one or more probes specific for nucleic acid of an Atopobium species, a Porphyromonas species, and/or a Peptoniphilus species can be designed and used to detect the presence of an Atopobium species, a Porphyromonas species, and/or a Peptoniphilus species within a sample with or without performing nucleic acid sequencing. For example, a PCR assay that includes a PCR primer pair designed to amplify nucleic acid of A. vaginae and not nucleic acid from other species can be used to detect the presence of A. vaginae within a sample. For example, a PCR assay that includes a PCR primer pair designed to amplify nucleic acid of P. somerae, or a species having 16S rRNA that is greater than 98 percent identical to a 16S rRNA sequence of P. somerae, and not nucleic acid from other species can be used to detect the presence of P. somerae, or a species having 16S rRNA that is greater than 98 percent identical to a 16S rRNA sequence of P. somerae, within a sample. For example, a PCR assay that includes a PCR primer pair designed to amplify nucleic acid of P. harei or P. coxii and not nucleic acid from other species can be used to detect the presence of P. harei or P. coxii within a sample.

In some cases, fluorescence in situ hybridization (FISH) can be performed to detect the presence of an Atopobium species, a Porphyromonas species, and/or a Peptoniphilus species within a sample. For example, FISH can be performed using one or more FISH probes designed to hybridize to nucleic acid of A. vaginae can be used to detect the presence of A. vaginae within a sample. For example, FISH can be performed using one or more FISH probes designed to hybridize to nucleic acid of P. somerae, or a species having 16S rRNA that is greater than 98 percent identical to a 16S rRNA sequence of P. somerae, can be used to detect the presence of P. somerae, or a species having 16S rRNA that is greater than 98 percent identical to a 16S rRNA sequence of P. somerae, within a sample. For example, FISH can be performed using one or more FISH probes designed to hybridize to nucleic acid of P. harei or P. coxii can be used to detect the presence of P. harei or P. coxii within a sample.

In some cases, a reproductive tract sample can be assessed using both a PCR-based technique and a FISH technique. In such cases, those samples where both the PCR-based technique and the FISH technique revealed the presence of an Atopobium species, a Porphyromonas species, and/or a Peptoniphilus species can be identified or classified as containing an Atopobium species, a Porphyromonas species, and/or a Peptoniphilus species, while those samples where both the PCR-based technique and the FISH technique revealed the absence of an Atopobium species, a Porphyromonas species, and/or a Peptoniphilus species, can be identified or classified as lacking an Atopobium species, a Porphyromonas species, and/or a Peptoniphilus species. In cases where only one of the two techniques revealed the presence of an Atopobium species, a Porphyromonas species, and/or a Peptoniphilus species, the sample optionally can be re-assessed and/or further analysis can be performed (e.g., sequencing).

In some cases, the presence or absence of an Atopobium species, a Porphyromonas species, and/or a Peptoniphilus species can be detected simultaneously. In some cases, the presence or absence of an Atopobium species, a Porphyromonas species, and/or a Peptoniphilus species can be detected sequentially, and in any order.

In some cases, female mammals having endometrial hyperplasia or endometrial cancer can have a high vaginal pH. For example, a high vaginal pH can be any pH greater than 4.5 (e.g., 4.6 or higher, 4.7 or higher, 4.8 or higher, 4.9 or higher, 5.0 or higher, 5.1 or higher, 5.2 or higher, 5.3 or higher, 5.4 or higher, or 5.5 or higher). Any appropriate method can be used to detect the pH of a reproductive tract sample. For example, pH paper can be used to determine pH of reproductive tract sample (e.g., a vaginal swab).

Once identified as having endometrial hyperplasia based at least in part on the presence of an Atopobium species (e.g., A. vaginae), a Porphyromonas species, and/or a Peptoniphilus species (e.g., P. harei or P. coxii) in the upper reproductive tract and the absence of an Atopobium species (e.g., A. vaginae), a Porphyromonas species, and/or a Peptoniphilus species (e.g., P. harei or P. coxii) within the lower reproductive tract, as described herein, a therapy (e.g., a hormone therapy) can be administered to a female mammal. For example, a female mammal identified as having endometrial hyperplasia as described herein can be treated by administering progesterone (e.g., cyclic or continuous progestin therapy). In some cases, a female mammal identified as having endometrial hyperplasia as described herein can be treated by surgical removal of the uterus (e.g., a vaginal hysterectomy, an abdominal hysterectomy, a total laparoscopic hysterectomy, a total hysterectomy with lateral or bilateral salpingo-oophorectomy, or a radical hysterectomy) of the female mammal in combination with or in place of a therapy.

Once identified as having endometrial cancer based at least in part on the presence of an Atopobium species (e.g., A. vaginae), a Porphyromonas species, and/or a Peptoniphilus species (e.g., P. harei or P. coxii) in the lower reproductive tract as described herein, a therapy (e.g., at least the uterus of the female mammal can be surgically removed) can be administered to a female mammal. For example, a female mammal identified as having endometrial cancer as described herein can be treated by performing a total hysterectomy (e.g., a vaginal hysterectomy, an abdominal hysterectomy, a total laparoscopic hysterectomy, a total hysterectomy with lateral or bilateral salpingo-oophorectomy, or a radical hysterectomy). In some cases, a female mammal identified as having endometrial cancer as described herein can be treated using radiation therapy (e.g., high-energy x-ray therapy), chemotherapy (e.g., paclitaxel, carboplatin, doxorubicin, and/or cisplatin therapy), or hormone therapy (e.g., tamoxifen, goserelin, leuprolide, exemestane, anastrozole, and/or letrozole therapy) in combination with or in place of surgery.

When treating a female mammal (e.g., a female human) having an endometrial cancer as described herein, the endometrial cancer can be Type I endometrial cancer or Type II endometrial cancer.

In some cases, a female mammal having endometrial hyperplasia based at least in part on the presence of an Atopobium species (e.g., A. vaginae), a Porphyromonas species, and/or a Peptoniphilus species (e.g., P. harei or P. coxii) in the upper reproductive tract and the absence of an Atopobium species (e.g., A. vaginae), a Porphyromonas species, and/or a Peptoniphilus species (e.g., P. harei or P. coxii) in the lower reproductive tract, or a female mammal having endometrial cancer based at least in part on the presence of an Atopobium species (e.g., A. vaginae), a Porphyromonas species, and/or a Peptoniphilus species (e.g., P. harei or P. coxii) in the lower reproductive tract can be treated with one or more agents (e.g., antibiotics) having the ability to kill Atopobium species, Porphyromonas species, and/or Peptoniphilus species. For example, a female mammal identified as having endometrial hyperplasia as described herein or a female mammal identified as having endometrial cancer as described herein can be treated by administering an antibiotic to the mammal under conditions wherein the number of Atopobium species bacteria, the number of Porphyromonas species bacteria, and/or the number of Peptoniphilus species bacteria present within the upper reproductive tract and/or lower reproductive tract are reduced.

In some cases, a female mammal having a likelihood of developing endometrial cancer (e.g., a female mammal having endometrial hyperplasia) and/or having endometrial cancer can be treated with one or more agents (e.g., antibiotics) having the ability to kill Atopobium species, Porphyromonas species, and/or Peptoniphilus species. For example, a female mammal identified as having endometrial hyperplasia and/or endometrial cancer can be treated by administering an antibiotic to the mammal under conditions wherein the number of Atopobium species bacteria, the number of Porphyromonas species bacteria and/or the number of Peptoniphilus species bacteria present within the reproductive tract are reduced. Examples of antibiotics that can be used to treat a female mammal as described herein include, without limitation, benzylpenicillin, tetracycline, amoxicillin, ampicillin, ticarcillin, piperacillin, cephalothin, cefuroxime, cefotaxime, cefoxitin, imipenem erythromycin, cefamandole, cephaloridine, oleandomycin, metronidazole, spiramycin, and clindamycin.

The invention will be further described in the following examples, which do not limit the scope of the invention described in the claims.

### EXAMPLES

### Example 1—Presence of a Porphyromonas Species and an Atopobium Species in the Reproductive Tract Indicates the Presence of Endometrial Hyperplasia or Endometrial Cancer

### Subject Enrollment

31 subjects were enrolled in this study. The inclusion criteria were the following: 18 years of age or older; females undergoing hysterectomy by any standard surgical approach; undergoing hysterectomy for benign disease, hyperplasia, or any stage of endometrial cancer. Patients with any of the following criteria were excluded from the study: women who were pregnant or nursing; taken antibiotics within 2 weeks preceding surgery, surgeon using morcellation during the hysterectomy procedure, due to the size of the uterus or for any other reason. Upon enrollment the subjects were requested to fill out an optional questionnaire about sexual and reproductive health and history. The metadata from the questionnaires was stored at REDCap (Harris et al., 2009. J. Biomedical Informatics 42: 377-381). Cancer subjects were also requested to provide a stool sample for the search for putative endometrial cancer signatures.

### Vaginal and Cervical Sample Collection

All subjects were requested not to douche with betadine on the surgery day or the day immediately preceding it. All the vaginal and cervical swabs and scrapes were collected by the surgeon (with guidance on site by the research team) immediately after the administration of anesthesia and immediately preceding the standard pre-surgical betadine douche. Both the vaginal and cervical swabs were performed with 3 sterile Dacron swabs each and placed in a sterile tube with 1 ml of Tris-EDTA (TE) buffer kept on dry ice until storage at −80° C. One of the vaginal swabs was used for immediate on-site vaginal pH measurement with a Hydrion measuring pH tape. The scrapes were performed using sterilized (autoclaved at 121° C. for 20 minutes) pap smear spatulas and placed in sterile tubes with TE buffer kept in dry ice until storage at −80° C.

### Uterine, Fallopian and Ovarian Sample Collection

Once removed, the uterus, fallopian tubes and ovaries were handed by the surgeon to the instrumentalist nurse who placed them inside a sterile transport bag and into a closed sterile container. The research team then transported the container to the pathology lab (within the same clean area) where the organs were handed to a pathology assistant (PA) to be processed under sterile conditions. The grossing station where the specimen was processed was sterilized by the research team, including all the tools needed by the PA for handling. The PA used surgical gloves and mask when handling the specimen. The PA performed a bilateral cut of the uterus and splayed it. The research team advanced to the collection of the uterine swabs (Dacron) and scrapes (sterilized pap smear spatulas) and documentation (by placement of push pins in sampled locations and digital photograph). The PA then proceeded to the aseptic collection of samples needed for the diagnosis, and once complete, the research team collected the uterine, fallopian and ovarian biopsies (approximately 4 mm of tissue was collected per biopsy by the use of a sterile tweezers, scalpels and surgical ruler). Each collected sample was placed in a sterile tube with 1 mL of TE buffer and kept on dry ice until storage at −80° C. A petri dish with LB was kept open on the grossing station during sample collection to detect any possible airborne contamination of the specimen. The LB was swabbed and the swab was stored in a tube with 1 mL of TE and kept on dry ice until storage along with all the other samples.

### Sample Processing

Once thawed the swab and scrape samples were vortexed to bring the collected material into solution. The biopsy samples were mushed by the use of sterile pestles. The swab and scrape samples were centrifuged for 10 minutes at 10,000 g to collect the bacterial cells, and the supernatant was discarded. All genomic DNA extractions were performed by using the MoBio PowerSoil Kit (MoBio Laboratories, Inc., Carlsbad, Calif.); however, instead of vortexing, MP FastPrep (MP Biomedicals, Solon, Ohio) for 60 seconds at 6.0 m/s was substituted to obtain a more effective and rapid lysis of the cells. After extraction the DNA content was measured using High Sensitivity Qubit (Life Technologies Corporation, Carlsbad, Calif.). The V3-V5 region of the 16S rDNA was then amplified through a polymerase chain reaction (PCR) as follows: 25 μl of Kapa HiFi (Kapa Biosystems, Woburn, Mass.), 1.5 μl (10 μM) forward primer, 1.5 μl (10 μM) reverse primer, 50 ng of DNA with the remaining volume being added by molecular grade water (up to a final volume of 50 μl per reaction). The forward primer was the universal primer 357F (5′GTCCTACGGGAGGCAGCAG3′; SEQ ID NO:1) with the added construct on the 5′ end of the 5′ Illumina Adapter (5′AATGATACGGCGACCACCGAGATCTACAC3′; SEQ ID NO:2) +Forward Primer Pad (5′ TATGGTAATT3′; SEQ ID NO:3) to a total sequence: 5′AATGATACGGCGACCACCGAGATCTACACTATGGTAATTGTCCTACGGGAG GCAGCAG3′ (SEQ ID NO:4) and the universal bacterial reverse primer was 926R (5′CCGTCAATTCMTTTRAGT3′; SEQ ID NO:5) with an added construct on the 5′ end of the reverse complement of 3′ Illumina adapter (5′CAAGCAGAAGACGGCATACGAGATGCCGCATTCGAT3′; SEQ ID NO:6) +Barcode (12 base pairs) to a total sequence: 5′CAAGCAGAAGACGGCATACGAGATGCCGCATTCGATXXXXXXXXXXXXCC GTCAATTCMTTTRAGT3′ (SEQ ID NO:7). The barcode introduced in the reverse primer construct was unique to each sample, functioning as a genetic ID for sequencing. The PCR cycle was the following: 95° C. for 3 minutes, 98° C. for 20 seconds, 70° C. for 15 seconds, 72° C. for 15 seconds, cycle repeated for 34 times and 72° C. for 5 minutes. The products of the amplification were verified by TapeStation D1K Tape (2200 TapeStation Instrument, Agilent Technologies, Santa Clara, Calif.) to be free of contamination and the expected amplification size, approximately 700 base pairs. If the amplification was unsuccessful, the parameters of the reaction or cycle were adjusted in repeated attempts. In some cases (mostly biopsy samples) the amplification was not successful even after repeated attempts. The reduced number of microorganisms present in the upper reproductive tract are likely to justify this result and attests for the success of the sterile collection of the samples. In samples that failed 16S rDNA amplification, NEBNext Microbiome DNA Enrichment Kit (New England Biolabs Inc., Ipswitch, Mass.) was used to separate the microbiome from the human DNA to increase the odds of a successful amplification from samples naturally enriched with human DNA (mostly tissue samples). Controls of both the DNA extraction and Microbiome Enrichment processes were performed and included taxa prominent in samples from the control cohort. Upon verification the PCR products were purified using Agencourt AMPure (Beckman Coulter, Brea, Calif.). After purification the concentrations were measured using Qubit High Sensitivity. The 16S sequencing was performed by the MGF (Medical Genome facility at Mayo Clinic, Rochester) using a high-throughput next-generation Illumina MiSeq (San Diego, Calif.) sequencing platform.

### Sequence Analysis

Sequence reads were aligned with our own custom multiple alignment tool known as the Illinois-Mayo Taxon Operations for RNA Dataset Organization (IM-TORNADO) that merges paired end reads into a single multiple alignment and obtains taxa calls (Sipos et al., 2010 PLoS One 5:e15220). IM-TORNADO then clusters sequences into operational taxonomic units (OTUs) using AbundantOTU+(Ye et al., 2011 Proceedings (IEEE Int Conf Bioinformatics Biomed) 2010:153-157).

### Sequencing Outcome

A total of 16,366,472 sequence reads (17,657 to 828,181 reads per sample) were obtained (mean of 199,591±190,153 reads) after quality control. Further processing for visualization was performed using QIIME (Caporaso et al. 2010) and METAGENassist (Arndt et al., 2012 Nucleic Acids Res. 40:W88-95).

### Data Analysis

α-diversity and β-diversity analysis. To compare the overall microbta between cohorts, we summarized microbiota data using both α-diversity and β-diversity. α-diversity reflects species richness and evenness within bacterial populations. Two α-diversity metrics, the observed OTU number and the Shannon index, were investigated. The observed OTU number reflects species richness, whereas the Shannon index measures both species richness and evenness. β-diversity reflects the shared diversity between bacterial populations in terms of ecological distance; different distance metrics provide distinctive views of community structure. Two β-diversity measures, unweighted and weighted UniFrac distances, were calculated using the OTU table and a phylogenetic tree (“GUniFrac” function in the R package GUniFrac) (Chen et al. 2012 Bioinformatics 28:2106-2113). The unweighted UniFrac reflects differences in community membership (i.e., the presence or absence of an OTU), whereas the weighted UniFrac captures this information and also differences in abundance. Rarefaction was performed on the OTU table before calculating the distances.

To assess the association with α-diversity, a linear mixed effects model (LME) to the α-diversity metrics with a random intercept was fitted for each subject (gm& function in R package ‘nlme’), adjusting for covariates if necessary. Wald test was used to assess the significance. To assess the association with β-diversity measures, a variant of PERMANOVA procedure (“adonis” function in the R ‘vegan’ package) was used, which is a multivariate analysis of variance based on distance matrices and permutation (McArdle and Anderson, 2001 Ecology 82:290-297). To retain the within-subject correlation, a block-permutation scheme was used, where samples from the same subject were assigned a different subject ID. Significance was assessed by 1,000 permutations, and covariate was adjusted if necessary. Ordination plots were generated using non-metric multidimensional scaling (NMDS) as implemented in R (“metaMDS” function in the R ‘vegan’ package).

To test for the correlation between organs, a permutation test based on Bray-Curtis distance was used with the test statistic calculated as the distance between the organs from different subjects minus the distance between the organs from the same subject. The subject IDs were permuted for the same organ type using the same block-permutation scheme as above. P value was calculated as the percentage of permutations that produce a test statistic more extreme than what is observed. To identify the taxa shared by both organs, a taxon-specific Euclidean distance was used, defined based on the presence and absence of a given taxon, and applied the same permutation test. To test whether the distance from cohort 1 to cohort 2 is greater than the distance from cohort 1 to cohort 3, a permutation test with the test statistic as the difference between these two distances was used, and block-permutation was used for assessing the significance.

Differential abundance analysis. A differential abundance analysis was conducted at phylum, family and genus levels, and filtered rare taxa with prevalence less than 20% to reduce the number of the tests. A generalized mixed-effects model was fit to the taxa count data using PQL method, assuming a random intercept for each subject to account for within-subject correlation (‘glmmPQL’ in R ‘MASS’ package). An overdispersed Poisson was fit to the counts if the zero proportion is less than 25%, and an overdispersed Binomial model (presence/absence) otherwise. For the overdispersed Poisson model, the log of library size was included as an offset to account for variable sequencing depth. In the overdispersed Binomial model, the log of library size was included as a covariate to account for potential dependence of occurrence probability with sequencing depth. The winsorized data (97% upper quantile) was used to reduce the potential impact of outliers upon the parameter estimates. To improve power to detect differential taxa, which show consistent change in both the uterus and lower tract microbiome, the uterus and lower tract data were pooled, and included the sampling site (uterus/lower tract) as a covariate in the model. The same analyses were also repeated for both data sets separately to confirm the source of the identified signals using pooled data.

Statistical significance was assessed based on Wald test. False discovery rate (FDR) control (B-H procedure, ‘p.adjust’ in standard R packages) was used for correcting for multiple testing, and FDR-adjusted p value or q values will be reported. All statistical analyses were performed in R 3.0.2 (R Development Core Team, Vienna, Austria). The ROC curve and AUC was generated by using the median of the replicates with the software generated by John's Hopkins (rad.jhmi.edu/jeng/javarad/rocaROCFITi.html).

### Results

Subject Population. A total of 31 Caucasian patients undergoing a hysterectomy were included in this study. Of those, 10 women were diagnosed with a benign gynecologic condition (control cohort), 4 women were diagnosed with endometrial hyperplasia (cancer precursor, hyperplasia cohort), and 17 women were diagnosed with endometrial cancer (cancer cohort). All diagnoses were made based on final surgical pathology following hysterectomy. Patients diagnosed with endometrial cancer were significantly older, predominantly postmenopausal and hypertensive (Table 1).

TABLE 1
Patient demographics.

| | | | | Hyperplasia | p-value vs | p-value vs |
|---|---|---|---|---|---|---|
| Variables | Benign (N = 10) | Cancer (N = 17) | p-value | (N = 4) | benign | cancer |
| Age (years) - Median, IQR | 44.5 (42.5-52.5) | 64 (58-71) | 0.0001 | 54 (50.75-62.5) | 0.0552 | 0.08 |
| Caucasian Ethnicity (%) | 10 (100) | 17 (100) | | 4 (100) | | |
| BMI - Median, IQR | 26.6 (23.8-34.1) | 32.1 (26.8-40.2) | 0.07 | 35.4 (24-40.8) | 0.29 | 0.89 |
| Menopausal status | | | 0.0034 | | >0.99 | 0.0526 |
| Pre/Peri | 8 | 3 | | 3 | | |
| Post | 2 | 14 | | 1 | | |
| Gravida - Median, IQR | 2 (2-3.25) | 1.5 (0-4) | 0.666 | 0 (0-2.25) | 0.1 | 0.19 |
| Parity - Median, IQR | 2 (2-3) | 1.5 (0-4) | 0.569 | 0 (0-2.25) | 0.13 | 0.23 |
| History of Hypertension | | | 0.0362 | | 0.85 | 0.31 |
| Yes | 1 | 10 | | 1 | | |
| No | 8 | 7 | | 3 | | |
| Unknown | 1 | 0 | | 0 | | |
| History Diabetes | | | 0.621 | | >0.99 | 0.54 |
| Yes | 1 | 4 | | 0 | | |
| No | 9 | 13 | | 4 | | |
| Smoking Status | | | 0.5911 | | 0.46 | 0.75 |
| Never smoker | 5 | 9 | | 3 | | |
| Previous smoker | 2 | 4 | | 1 | | |
| Current smoker | 3 | 2 | | 0 | | |
| Unknown | 0 | 2 | | 0 | | |
| Vaginal pH | | | 0.0053 | | >0.99 | 0.07 |
| Normal | 6 | 1 | | 2 | | |
| High | 4 | 15 | | 2 | | |
| Unknown | 0 | 1 | | 0 | | |
| Histotype (%) | | | | | | |
| Endometrioid Adenocarcinoma | — | 11 (64.7) | | — | | |
| Serous Adenocarcinoma | — | 3 (17.6) | | — | | |
| Mucinous Adenocarcinoma | — | 1 (5.9) | | — | | |
| Squamous adenocarcinoma | — | 1 (5.9) | | — | | |
| Carcinosarcoma | — | 1 (5.9) | | — | | |
| Grade (%) | | | | | | |
| G1/G2 | — | 13 (76.5) | | — | | |
| G3 | — | 4 (23.5) | | — | | |
| Stage (%) | | | | | | |
| I | — | 13 (76.5) | | — | | |
| III/IV | — | 4 (23.5) | | — | | |

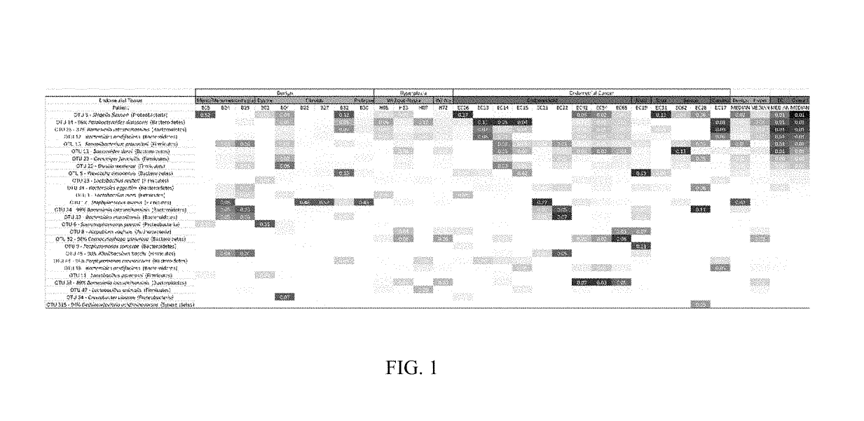

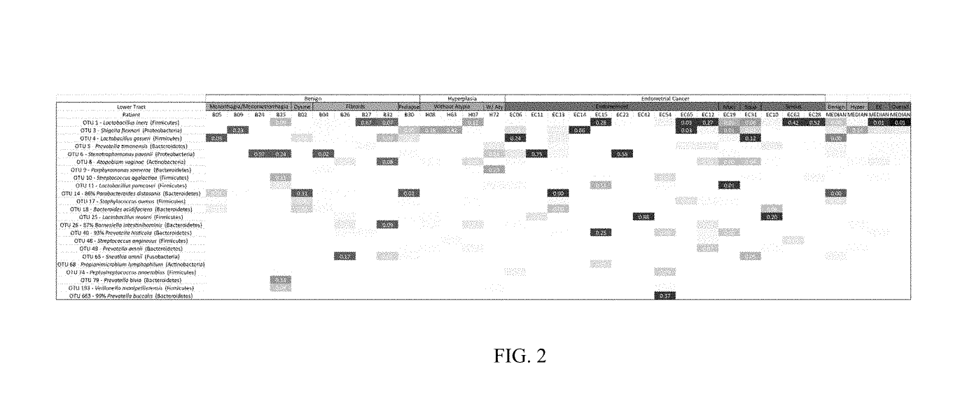

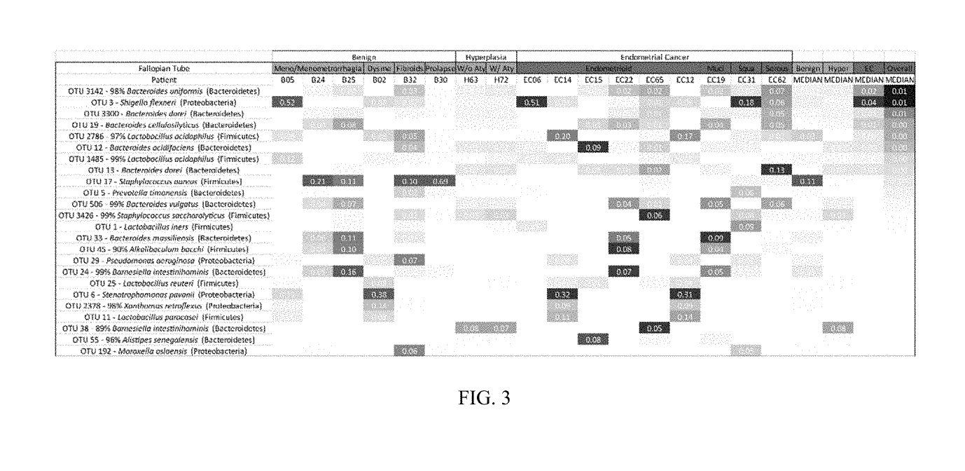

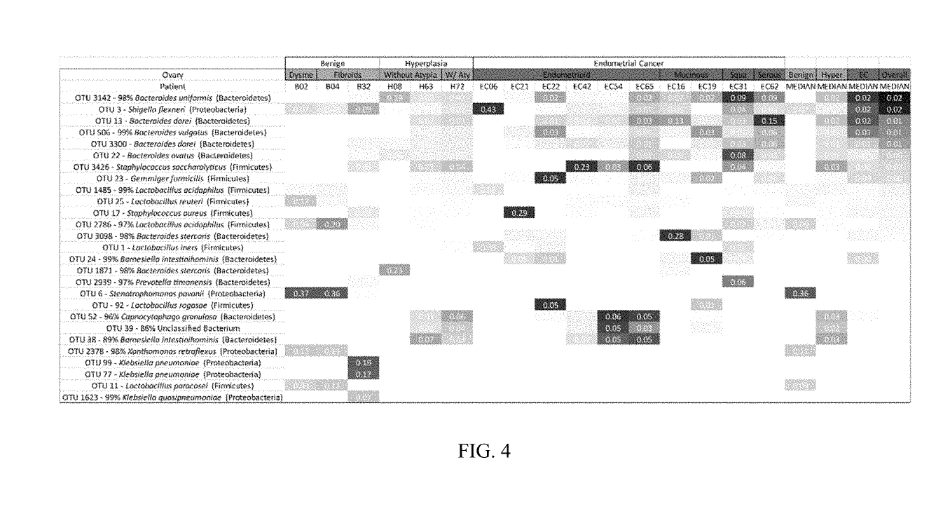

Microbiome Characterization. In order to characterize the microbiome of the patients vaginal and cervical samples (lower tract) were collected in the operating room and endometrial, fallopian and ovarian samples were collected in the pathology laboratory (see Methods section). The deep-sequencing of the V3-V5 16S rDNA region of all 238 collected samples resulted in the identification of 3,545 Operational Taxonomic Units (OTUs). The endometrial microbiome was dominated by Shigella and Barnesiella, with Staphylococcus, Blautia and Parabacteroides particularly relevant in the benign cohort and Bacteroides and Faecalibacterium more relevant in the endometrial cancer cohort (FIG. 1). The uterine microbiome results were consistent with the very limited number of studies that have assessed the human microbiome composition through culture-based methods, where Escherichia, Streptococcus, Staphylococcus and Enterococcus were found to be the predominant taxa in women with chronic endometritis and dysfunctional bleeding (Cicinelli et al., 2008 Fertility and sterility 89:677-684). The very recent 16S rDNA assessment of the uterine microbiome via transcervical collection was also consistent with Bacteroides being a dominant uterine taxa (Verstraelen et al., 2015 “Characterisation of the human uterine microbiome in non-pregnant women through deep sequencing of the 16S rRNA gene,” PeerJ PrePrints). In the lower tract (vagina and cervix), Prevotella and Lactobacillus were the dominant taxa, with Stenotrophomonas and Shigella more characteristic in the benign cohort, and Porphyromonas more common in the endometrial cancer cohort (FIG. 2). These results were consistent with the general profiles reported by others (Ravel et al., 2011 Proceedings of the National Academy of Sciences 108:4680-4687; and Brotman et al., 2014 Menopause (New York, N.Y.) 21:450-458), with the exception of Stenotrophomonas. Because the benign population in this study was not gynecologically healthy, but instead presented with a variety of conditions (pelvic pain, abnormal bleeding, fibroids, and prolapse), it is possible that Stenotrophomonas may be more prominent in this patient population than in an asymptomatic group of subjects. Although it is also possible that this could be the result of contamination, this taxon was not prominent in our controls, therefore it is unlikely that this is the case. In the Fallopian tubes, Shigella and Bacteroides were the most dominant taxa, with Staphylococcus, Lactobacillus, Barnesiella and Pseudomonas commonly appearing in the benign cohort (FIG. 3). In the ovaries, Stenotrophomas, Xanthomonas and Lactobacillus dominated the benign cohort, while Bacteroides dominated the endometrial cancer cohort (FIG. 4). There is no current literature on the human microbiome composition of Fallopian tubes or ovaries.

Organ Microbiome Collection. The microbiome between the different organs was correlated. For instance, whether the vaginal microbiome of a given patient resembled the uterine microbiome of that particular patient more than the uterine microbiome of any other patient. The results showed a very significant correlation between all organs based on a distance-based permutation test (see Methods and Table 2).

Surprisingly, the correlation was also significant, though to a lesser degree, for the stool samples when compared to all organs. The correlation structure holds for both benign and cancer cohorts (Table 3). Genus level analysis revealed several genera that were significantly shared between lower tract and uterus (Table 4). These results are indicative of an overall host specific microbiome effect and/or transfer of microbiomes across the different organs. The correlation between organs also suggests a potential gain in statistical power by a combined analysis. Both combined (uterus+lower tract) and separate analyses were performed when comparing the microbiota between different disease states.

TABLE 3
Organ correlation with benign and endometrial cancer patients.

| | | | | |
|---|---|---|---|---|
| FALLO- | | | | |
| PIAN | LOWER | OVARY | STOOL | UTERUS |

Benign

| | | | | | |
|---|---|---|---|---|---|
| FALLOPIAN | 0 | 0.048 | 0.053 | NA | 0.001 |
| LOWER | 0.048 | 0 | 0.009 | NA | 0.045 |
| OVARY | 0.053 | 0.009 | 0 | NA | 0.026 |
| STOOL | NA | NA | NA | 0 | NA |
| UTERUS | 0.001 | 0.045 | 0.026 | NA | 0 |

Cancer

| | | | | | |
|---|---|---|---|---|---|
| FALLOPIAN | 0 | 0.019 | 0.001 | 0.018 | 0.001 |
| LOWER | 0.019 | 0 | 0.001 | 0.034 | 0.001 |
| OVARY | 0.001 | 0.001 | 0 | 0.04 | 0.001 |
| STOOL | 0.018 | 0.034 | 0.04 | 0 | 0.026 |
| UTERUS | 0.001 | 0.001 | 0.001 | 0.026 | 0 |

*Bray-Curtis distance is used with 1,000 permutations.

TABLE 4
Genera correlation between lower tract and uterus.

| | p | |
|---|---|---|
| | value | q value |
| Actinobacteria; Actinobaculum | 0.07 | 0.27363636 |
| Actinobacteria; Actinomyces | 0.258 | 0.43496154 |
| Actinobacteria; Adlercreutzia | 0.499 | 0.58689189 |
| Actinobacteria; Amycolatopsis | 0.001 | 0.02866667 |
| Actinobacteria; Atopobium | 0.005 | 0.06142857 |
| Actinobacteria; Collinsella | 0.466 | 0.5805 |
| Actinobacteria; Coriobacterium | 0.72 | 0.73714286 |
| Actinobacteria; Corynebacterium | 0.572 | 0.64332468 |
| Actinobacteria; Mobiluncus | 0.251 | 0.43496154 |
| Actinobacteria; Propionibacterium | 0.382 | 0.50806154 |
| Bacteroidetes; Bacteroides | 0.486 | 0.5805 |
| Bacteroidetes; Butyricimonas | 0.363 | 0.50806154 |
| Bacteroidetes; Cellulophaga | 0.48 | 0.5805 |
| Bacteroidetes; Elizabethkingia | 0.373 | 0.50806154 |
| Bacteroidetes; Myroides | 0.381 | 0.50806154 |
| Bacteroidetes; Odoribacter | 0.465 | 0.5805 |
| Bacteroidetes; Parabacteroides | 0.14 | 0.381625 |
| Bacteroidetes; Pedobacter | 0.081 | 0.29741667 |
| Bacteroidetes; Porphyromonas | 0.147 | 0.38309091 |
| Bacteroidetes; Prevotella | 0.423 | 0.55118182 |
| Bacteroidetes; unclassified | 0.898 | 0.898 |
| Bacteroidetes; RC22 | 0.004 | 0.06142857 |
| Chrysiogenetes; Desulfurispirillum | 0.102 | 0.33738462 |
| Firmicutes; 1-68 | 0.263 | 0.43496154 |
| Firmicutes; Allobaculum | 0.001 | 0.02866667 |
| Firmicutes; Anaerococcus | 0.036 | 0.19915789 |
| Firmicutes; Anaerofilum | 0.208 | 0.416 |
| Firmicutes; Anaerostipes | 0.022 | 0.172 |
| Firmicutes; Anaerotruncus | 0.132 | 0.3784 |
| Firmicutes; Blautia | 0.092 | 0.31648 |
| Firmicutes; Butyrivibrio | 0.433 | 0.55579104 |
| Firmicutes; Christensenella | 0.187 | 0.416 |
| Firmicutes; Clostridium | 0.226 | 0.42085106 |
| Firmicutes; Coprobacillus | 0.293 | 0.44961404 |
| Firmicutes; Coprococcus | 0.128 | 0.3784 |
| Firmicutes; Dialister | 0.228 | 0.42085106 |
| Firmicutes; Dorea | 0.23 | 0.42085106 |
| Firmicutes; Enterococcus | 0.207 | 0.416 |
| Firmicutes; Epulopiscium | 0.719 | 0.73714286 |
| Firmicutes; Ethanoligenens | 0.505 | 0.58689189 |
| Firmicutes; Eubacterium | 0.022 | 0.172 |
| Firmicutes; Faecalibacterium | 0.523 | 0.59970667 |
| Firmicutes; Finegoldia | 0.13 | 0.3784 |
| Firmicutes; Lachnobacterium | 0.371 | 0.50806154 |
| Firmicutes; Lachnospira | 0.178 | 0.416 |
| Firmicutes; Lactobacillus | 0.003 | 0.06142857 |
| Firmicutes; Lutispora | 0.287 | 0.44876364 |
| Firmicutes; Megasphaera | 0.152 | 0.38447059 |
| Firmicutes; Moryella | 0.005 | 0.06142857 |
| Firmicutes; Oribacterium | 0.576 | 0.64332468 |
| Firmicutes; Oscillospira | 0.486 | 0.5805 |
| Firmicutes; Pediococcus | 0.042 | 0.19915789 |
| Firmicutes; Peptococcus | 0.083 | 0.29741667 |
| Firmicutes; Peptoniphilus | 0.02 | 0.172 |
| Firmicutes; Peptostreptococcus | 0.278 | 0.44592593 |
| Firmicutes; ph2 | 0.298 | 0.44961404 |
| Firmicutes; Phascolarctobacterium | 0.688 | 0.72156098 |
| Firmicutes; Pseudobutyrivibrio | 0.206 | 0.416 |
| Firmicutes; Roseburia | 0.325 | 0.47372881 |
| Firmicutes; Ruminococcus | 0.674 | 0.71560494 |
| Firmicutes; Shuttleworthia | 0.034 | 0.19915789 |
| Firmicutes; SMB53 | 0.384 | 0.50806154 |
| Firmicutes; Staphylococcus | 0.041 | 0.19915789 |
| Firmicutes; Streptococcus | 0.108 | 0.344 |
| Firmicutes; unclassified | 0.142 | 0.381625 |
| Firmicutes; Veillonella | 0.79 | 0.79929412 |
| Firmicutes; WAL_1855D | 0.242 | 0.43358333 |
| Fusobacteria; Fusobacterium | 0.253 | 0.43496154 |
| Fusobacteria; Sneathia | 0.009 | 0.09675 |
| Fusobacteria; Streptobacillus | 0.058 | 0.23752381 |
| Proteobacteria; Achromobacter | 0.28 | 0.44592593 |
| Proteobacteria; Acinetobacter | 0.028 | 0.19915789 |
| Proteobacteria; Agrobacterium | 0.65 | 0.69875 |
| Proteobacteria; Brevundimonas | 0.215 | 0.42022727 |
| Proteobacteria; Campylobacter | 0.051 | 0.2193 |
| Proteobacteria; Delftia | 0.206 | 0.416 |
| Proteobacteria; Erwinia | 0.591 | 0.65161538 |
| Proteobacteria; Klebsiella | 0.167 | 0.41034286 |
| Proteobacteria; Nautilia | 0.041 | 0.19915789 |
| Proteobacteria; Pseudomonas | 0.192 | 0.416 |
| Proteobacteria; Salmonella | 0.322 | 0.47372881 |
| Proteobacteria; Stenotrophomonas | 0.032 | 0.19915789 |
| Proteobacteria; Sutterella | 0.63 | 0.68582278 |
| Proteobacteria; unclassified | 0.044 | 0.19915789 |
| Spirochaetes; Treponema | 0.001 | 0.02866667 |
| Tenericutes; unclassified | 0.207 | 0.416 |

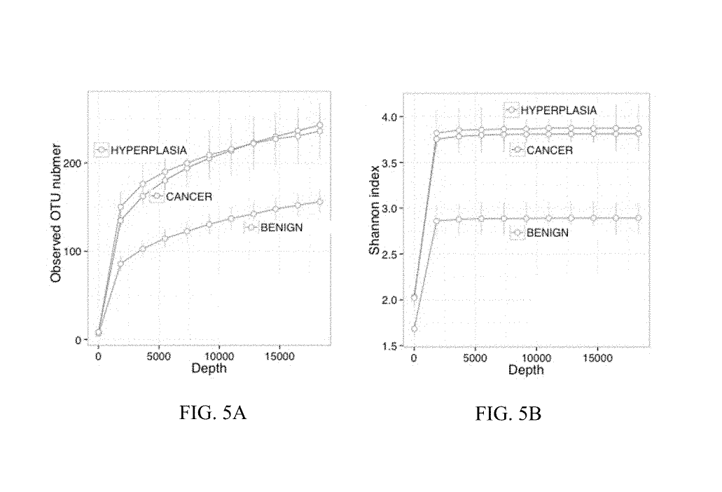

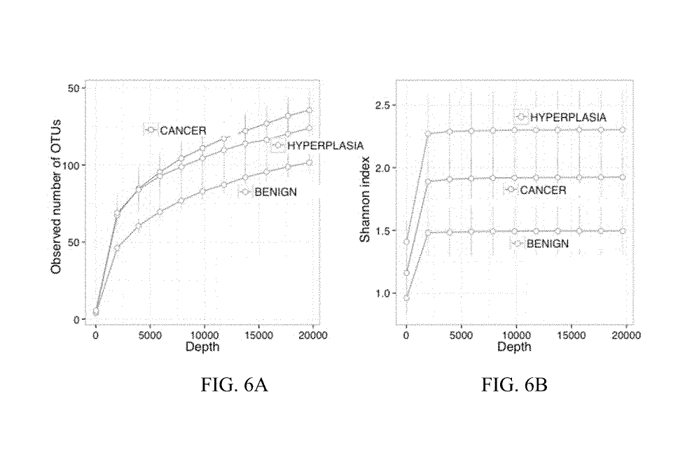

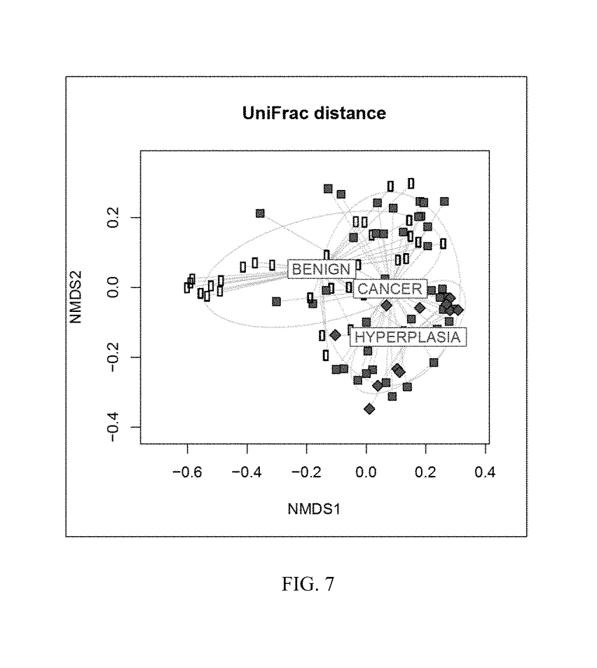

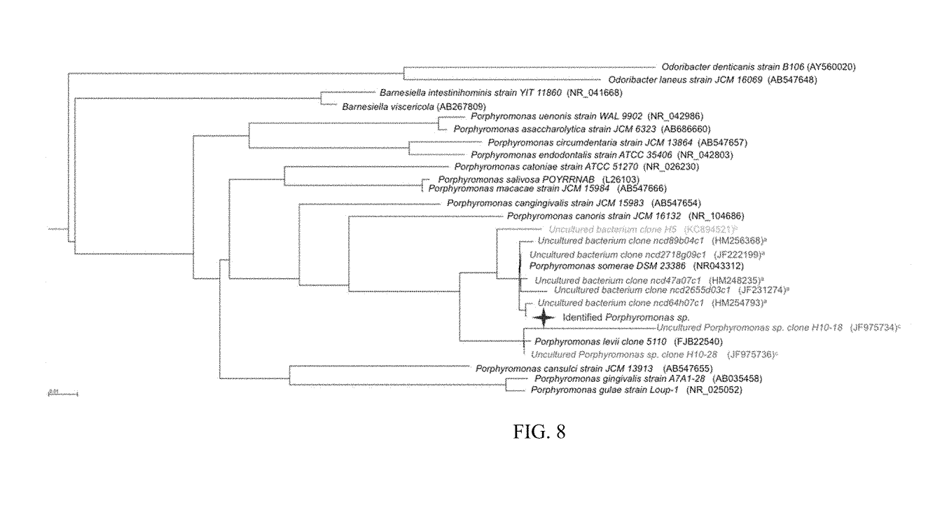

Overall Microbiome Structure Difference between Benign, Hyperplasia, and Endometrial Cancer. The overall microbiota structure between disease states were compared by investigating the α-diversity and β-diversity of the microbiota. The α-diversity (number of observed OTUs and Shannon index) in the cancer cohort was significantly higher than the benign cohort (p=0.003 and 0.01 for the two α-diversity metrics, LME) and the difference was much stronger in uterus (p=0.03 and 0.01, FIG. 5) than in the lower tract (p=0.17 and 0.31, FIG. 6). The endometrial α-diversity of the hyperplasia cohort was similar to the cancer cohort, and was also significantly higher than the benign cohort (p=0.07 and 0.04, FIG. 5). β-diversity analysis revealed a significant difference in the overall microbiota structure between the three cohorts (p=0.01, unweighted UniFrac, PERMANOVA, FIG. 7). Consistent with the α-diversity analysis, the difference was mainly observed in the uterus (p=0.05 and 0.11 for uterus and lower tract, unweighted UniFrac). Pairwise comparisons were conducted using the endometrial samples. The endometrial microbiome of both endometrial cancer and hyperplasia cohorts displayed some level of difference from the benign cohort (p=0.09 and 0.07, unweighted UniFrac). In contrast, the hyperplasia cohort was not very distinguishable from the endometrial cancer cohort (p=0.23, unweighted UniFrac) (FIG. 7). Comparison of the distance between benign and hyperplasia cohort to the distance between cancer and hyperplasia cohort revealed that the hyperplasia cohort was closer to the cancer cohort (p=0.05, unweighted UniFrac, permutation test, (FIG. 8). Interestingly, the distance between benign and hyperplasia cohort was also significantly larger than that between benign and cancer cohort (p=0.05, unweighted UniFrac, FIG. 8). Because endometrial hyperplasia can be a clinical precursor to endometrial cancer, and the uterine microbiome of the 4 patients diagnosed with endometrial hyperplasia was distinct from the benign cohort and presents some but not complete clustering with an endometrial cancer subgroup, these patients were removed in establishing an endometrial cancer microbiota signature. This allowed comparison of the benign and endometrial cancer cohorts without the impact of the hyperplasia cases. These were later introduced in secondary analysis.

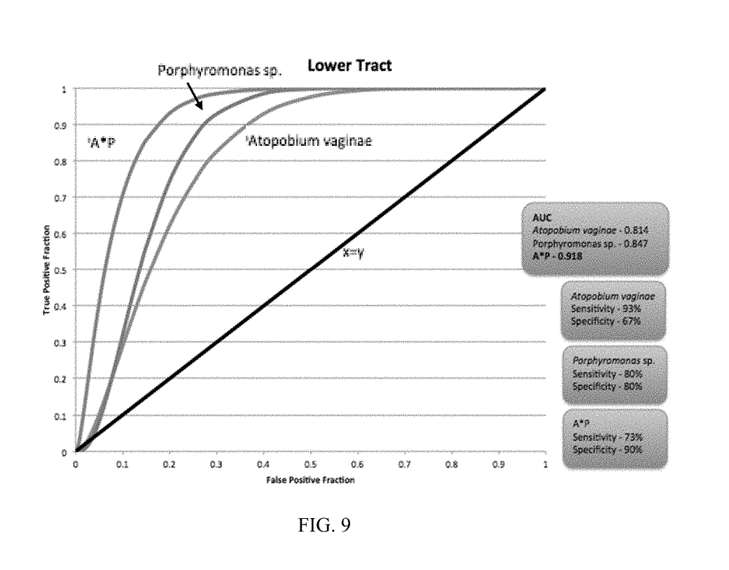

The data set also contains samples from fallopian tubes and ovaries. The microbiota were tested for differences between benign and cancer cohorts for these two organs. Interestingly, a significant difference for the ovaries (p=0.003, unweighted UniFrac, FIG. 9) suggested a connection between the ovarian microniche and endometrial cancer presence/absence.

Endometrial Cancer Microbiome Signature. After the overall microbiome assessment, taxa analyses were performed to determine whether the benign and endometrial cancer cohort displayed differential microbiota. A combined analysis was performed pooling the samples from both uterus and lower tract. At the genus level there were 12 taxa significantly enriched in the endometrial cancer cohort (Table 5 and FIG. 6, q<0.10). At a finer level (Operational Taxonomic Unit—OTU), 8 OTUs significantly associated with endometrial cancer (Table 6, q<0.05). OTU 8 (Atopobium sp.) and OTU 9 (Porphyromonas sp.) became of particular relevance since they were relatively highly abundant and pervasive across samples recovered from endometrial cancer patients and largely absent from the samples recovered in from patients in the benign cohort. The Atopobium V3-V5 16S rDNA signature matches (100%) that of Atopobium vaginae (Marconi et al., 2012 J. Lower Genital Tract Disease 16:127-132). The Porphyromonas signature is a close match (99% sequence identity) to P. somerae (FIG. 8), a described pathogen recovered from soft tissue and bone infections (Summanen et al., 2005 J. Clinical Microbiology 43: 4455-4459). Separate analyses of uterus and lower tract samples revealed a high concordance of the identified genera from the pooled analysis, indicating both uterus and lower tract microbiota may be informative for cancer diagnosis (Table 5).

TABLE 5
Significant bacterial genera between benign and endometrial cancer cohorts in
the vaginal, cervical and endometrial microbiome as determined by mixed effect model
(false discovery rate q < 0.10). All genera are enriched in the endometrial cancer cohort.

| | Combined q < 0.10 | | | | Uterus | | Lower | | |
|---|---|---|---|---|---|---|---|---|---|
| | Estimate* | SE | P value | Q value | Estimate* | P value | Estimate* | P value | Test |
| Firmicutes; Anaerostipes | 3.43 | 0.79 | 2.00E−04 | 0.0168 | 2.83 | 0.004 | 5.07 | 0.013 | Presence/absence |
| Firmicutes; ph2 | 3.08 | 0.83 | 1.00E−03 | 0.0308 | 28.05 | 1.000 | 3.64 | 0.012 | Presence/absence |
| Spirochaetes; Treponema | 3.94 | 1.07 | 1.10E−03 | 0.0308 | 2.81 | 0.016 | 30.05 | 1.000 | Presence/absence |
| Actinobacteria; Atopobium | 2.49 | 0.71 | 1.70E−03 | 0.0357 | 2.55 | 0.006 | 3.53 | 0.014 | Presence/absence |
| Bacteroidetes; Bacteroides | 1.11 | 0.33 | 2.60E−03 | 0.0437 | 1.01 | 0.005 | 1.21 | 0.012 | Counts |
| Proteobacteria; Arthrospira | 3.60 | 1.15 | 4.40E−03 | 0.0616 | 27.71 | 1.000 | 3.58 | 0.017 | Presence/absence |
| Firmicutes; Dialister | 1.21 | 0.40 | 6.10E−03 | 0.0732 | 0.93 | 0.112 | 1.52 | 0.023 | Presence/absence |
| Firmicutes; Peptoniphilus | 1.44 | 0.49 | 7.40E−03 | 0.0747 | 1.66 | 0.021 | 1.62 | 0.077 | Presence/absence |
| Firmicutes; 1-68 | 1.34 | 0.46 | 8.00E−03 | 0.0747 | 1.28 | 0.053 | 2.69 | 0.028 | Presence/absence |
| Firmicutes; Ruminococcus | 0.88 | 0.32 | 1.09E−02 | 0.0819 | 0.69 | 0.032 | 0.87 | 0.051 | Counts |
| Bacteroidetes; Porphyromonas | 1.82 | 0.66 | 1.11E−02 | 0.0819 | 1.56 | 0.051 | 2.92 | 0.029 | Presence/absence |
| Firmicutes; Anaerotruncus | 1.30 | 0.48 | 1.17E−02 | 0.0819 | 1.41 | 0.056 | 1.68 | 0.066 | Presence/absence |

*For test based on presence/absence, the estimate is interepreted as log odds ratio.
**For test based on counts, the estimate is interpreted as log fold change.

TABLE 6
Significant bacterial operational taxonomic units (OTUs) between benign and
endometrial cohorts in the vaginal, cervical and endometrial microbiome as determined
by mixed effect model (false discovery rate q < 0.05). All OTUs are enriched in the
endometrial cancer cohort.

| Combined q < 0.05 | Estimate* | SE | P value | Q value | Test |
|---|---|---|---|---|---|
| OTU 107: Firmicutes; Anaerostipes | 3.31 | 0.725 | 1.00E−04 | 0.014 | Presence/absence |
| OTU 143: Firmicutes; Ruminococcus | 3.08 | 0.738 | 3.00E−04 | 0.019 | Presence/absence |
| OTU 8: Actinobacteria; Atopobium | 2.48 | 0.603 | 4.00E−04 | 0.019 | Presence/absence |
| OTU 3197: Bacteroidetes; Bacteroides | 2.52 | 0.655 | 7.00E−04 | 0.024 | Presence/absence |
| OTU 3213: Bacteroidetes; Bacteroides | 1.74 | 0.499 | 1.80E−03 | 0.043 | Presence/absence |
| OTU 9: Bacteroidetes; Porphyromonas | 1.90 | 0.554 | 2.10E−03 | 0.043 | Presence/absence |
| OTU 138: Bacteroidetes; Bacteroides | 1.74 | 0.517 | 2.40E−03 | 0.043 | Presence/absence |
| OTU 181: Firmicutes; Dialister | 1.97 | 0.585 | 2.50E−03 | 0.043 | Presence/absence |

*The estimate is interepreted as log odds ratio

Vaginal pH and Endometrial Cancer. Vaginal pH was significantly correlated with an endometrial cancer diagnosis (p=0.0053), with endometrial cancer patients typically displaying a high vaginal pH (>5). However, the vaginal pH is known to raise in approximately 95% of postmenopausal women (Freedman, 2008 Menopause Management 17:9-13.) due to physiological and microbiological changes (Farage and Maibach, 2006 Archives of Gynecology and Obstetrics 273:195-202), so the correlation could not be detangled from age effects alone. Nevertheless, it was determined that the microbiome pH effects were independent of the microbiome disease effects in the uterus since the vaginal pH level was not significantly correlated with the uterine microbiome (p=0.22 and 0.29, unweighted and weighted UniFrac, PERMANOVA), indicating that they can be used as distinct factors.

Lower Tract Endometrial Cancer Biomarker. In the lower tract the association of Atopobium vaginae and the identified Porphyromonas sp. with a diagnosis of endometrial cancer predicted disease status (FIG. 9; sensitivity—73-93%, specificity—67-90%). The sensitivity is improved if vaginal pH is factored in, although specificity is decreased (Table 7; sensitivity—100%, specificity—60%).

Marginal PERMANOVA tests on the uterus samples revealed that these variables had less significant effects on the endometrial microbiota than the cohort effect and, though adjustment of these variables reduced the significance of the cohort effects due to correlation, the PERMANOVA p values were still on the low side, indicating that the observed difference could not be completely explained by these potential confounders.

Endometrial Hyperplasia Microbiome. 4 patients had a final diagnosis of endometrial hyperplasia, which is a known endometrial cancer precursor, in particular in the case of complex hyperplasia with atypia. Three patients had simple hyperplasia without atypia (H07, H08, and H63), and one had complex hyperplasia with atypia (H72). Interestingly, the Atopobium vaginae and the Porphyromonas sp. presence/absence profile of the vaginal microbiome of these 4 patients more closely resembled a benign microbiome signature (Table 7), while the uterine microbiome signature of two of them (H63 and H72) were closer to an endometrial cancer signature.

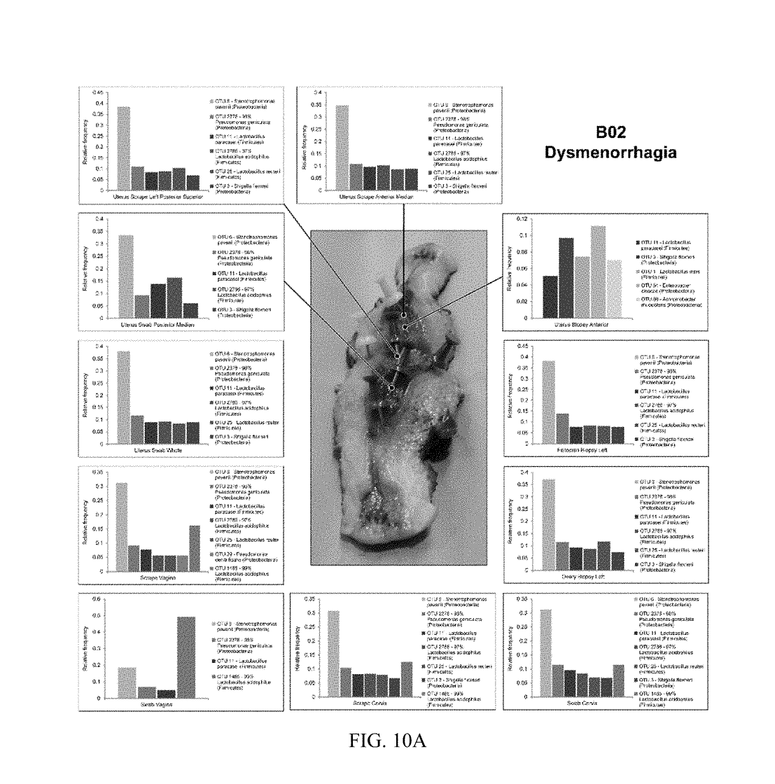

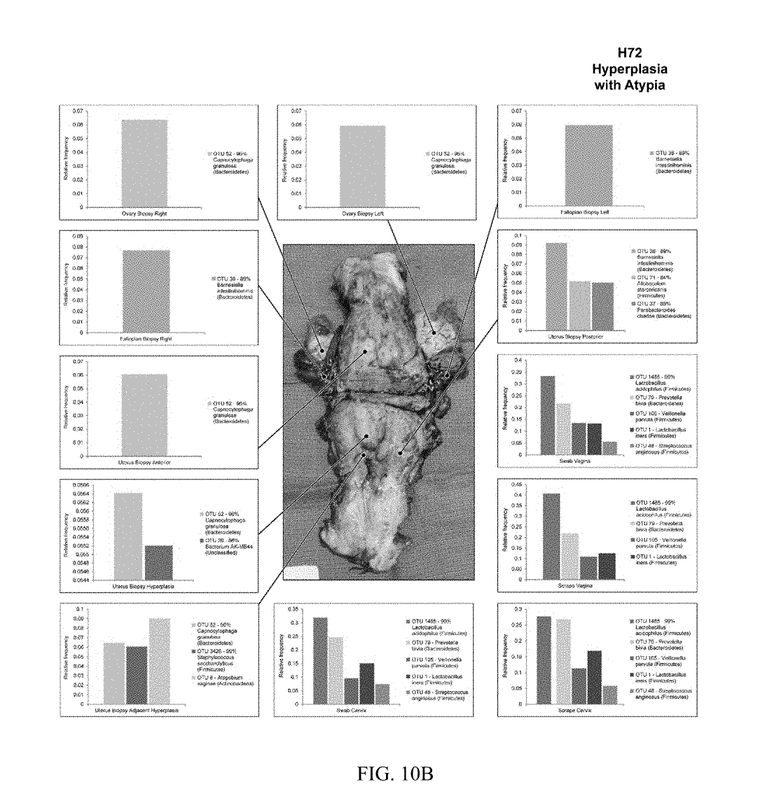

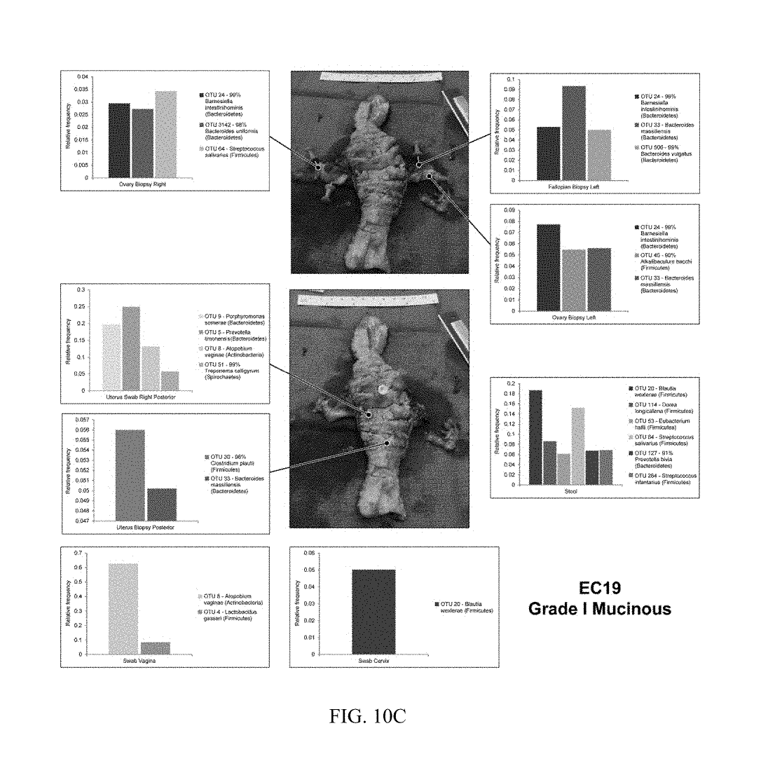

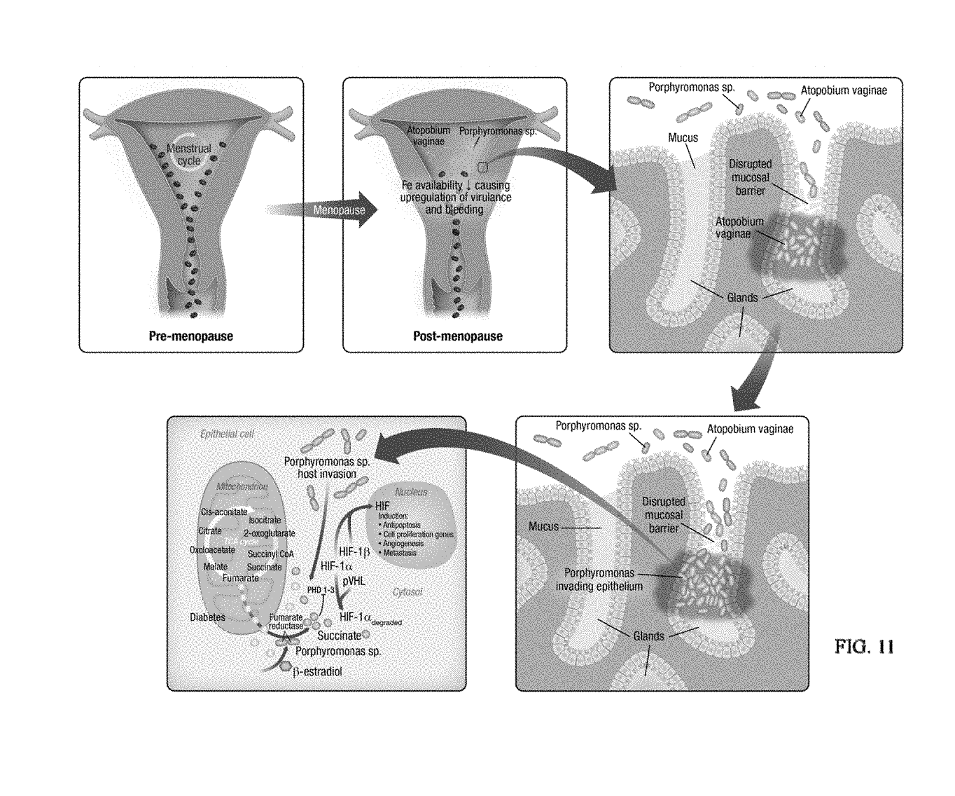

Snapshots of Progression. The correlation and variation between the microbiomes recovered is illustrated in the snapshots, which demonstrate the variable microbiome landscape within and between patients (FIG. 10). Bacterial DNA from 94% of the lower tract (vaginal/cervical) samples was successfully amplified, 87% uterine samples, 50% Fallopian, 61% ovarian, 29% urine and 17% peritoneal or ascites samples. This progression likely represented the bacterial burden in the different body sites.

These results demonstrated that endometrial hyperplasia and endometrial cancer can be detected by the presence of a Porphyromonas species and an Atopobium species in the reproductive tract. For example, the presence of a Porphyromonas species and an Atopobium species within the upper reproductive tract and the absence of a Porphyromonas species and an Atopobium species (e.g., A. vaginae) within the lower reproductive tract indicated that the female mammal has endometrial hyperplasia, while detecting the presence of a Porphyromonas species and an Atopobium species within the lower reproductive tract indicated that the female mammal has endometrial cancer.

### Example 2—Treating Endometrial Hyperplasia

An upper and lower reproductive tract sample is obtained from a female human, who is between about 25 and about 80 years old (e.g., between about 30 and about 65 years old, between about 35 and about 60 years old, or between about 40 and about 55 years old) and who lacks symptoms of endometrial hyperplasia as observable from a routine physical examination. The obtained sample is examined for the presence of an Atopobium species such as A. vaginae and the presence of a Porphyromonas species such as P. somerae or a Porphyromonas species having greater than 98 percent identity (e.g., greater than 99 percent identity) to P. somerae. In some cases, a PCR-based assay is performed to detect the presence of an Atopobium species and a Porphyromonas species. If an Atopobium species and a Porphyromonas species such as P. somerae or a Porphyromonas species having greater than 98 percent identity (e.g., greater than 99 percent identity) to P. somerae are detected in an upper reproductive tract sample, but not in a lower reproductive tract sample, then the female human is treated by performing hormone therapy (e.g., progesterone therapy such as cyclic or continuous progestin therapy). Alternatively or in addition, a hysterectomy can be performed.

### Example 3—Treating Endometrial Cancer

An upper or lower reproductive tract sample is obtained from a female human, who is between about 25 and about 80 years old (e.g., between about 30 and about 65 years old, between about 35 and about 60 years old, or between about 40 and about 55 years old) and who lacks symptoms of endometrial cancer as observable from a routine physical examination. The obtained sample is examined for the presence of an Atopobium species such as A. vaginae, and optionally for the presence of a Porphyromonas species such as P. somerae or a Porphyromonas species having greater than 98 percent identity (e.g., greater than 99 percent identity) to P. somerae. In some cases, a PCR-based assay is performed to detect the presence of an Atopobium species, and optionally a Porphyromonas species. If an Atopobium species such as A. vaginae, and optionally a Porphyromonas species such as P. somerae or a Porphyromonas species having greater than 98 percent identity (e.g., greater than 99 percent identity) to P. somerae, are detected in a lower reproductive tract sample, then the female human is treated by performing a hysterectomy (e.g., a vaginal hysterectomy, an abdominal hysterectomy, a total laparoscopic hysterectomy, a total hysterectomy with lateral or bilateral salpingo-oophorectomy, or a radical hysterectomy). Optionally, an endometrial biopsy, dilatation and curettage procedure, transvaginal ultrasound examination, or a combination thereof is performed prior to the hysterectomy to confirm that the female human has endometrial cancer.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating a female mammal having endometrial cancer, wherein said method comprises:
(a) identifying said mammal as having an Atopobium species and/or a Peptoniphilus species within the lower reproductive tract of said mammal, and
(b) removing at least the uterus of said mammal.

2. The method of claim 1, wherein said mammal is a human.

3. The method of claim 1, wherein said Atopobium species is A. vaginae.

4. The method of claim 1, wherein said Peptoniphilus species is P. harei or P. coxii.

5. The method of claim 1, further comprising identifying said mammal as having a Porphyromonas species within the lower reproductive tract of said mammal.

6. The method of claim 4, wherein said Porphyromonas species is P. somerae.

7. The method of claim 4, wherein said Porphyromonas species is a species having 16S rRNA that is greater than 98 percent identical to a 16S rRNA sequence of P. somerae.

8. A method for identifying a female mammal as having endometrial cancer, wherein said method comprises:
(a) detecting the presence of an Atopobium species and/or a Peptoniphilus species within the lower reproductive tract of said mammal, and
(b) classifying said mammal as having endometrial cancer.

9. The method of claim 8, wherein said mammal is a human.

10. The method of claim 8, wherein said Atopobium species is A. vaginae.

11. The method of claim 8, wherein said Peptoniphilus species is P. harei or P. coxii.

12. The method of claim 8, further comprising detecting the presence of a Porphyromonas species within the lower reproductive tract of said mammal.

13. The method of claim 11, wherein said Porphyromonas species is P. somerae.

14. The method of claim 11, wherein said Porphyromonas species is a species having 16S rRNA that is greater than 98 percent identical to a 16S rRNA sequence of P. somerae.

15. A method for treating a female mammal having endometrial hyperplasia, wherein said method comprises:
(a) identifying said mammal as having an Atopobium species, a Porphyromonas species, and/or a Peptoniphilus species within the upper reproductive tract of said mammal, but not within the lower reproductive tract of said mammal, and
(b) administering progestin therapy to said mammal.

16. The method of claim 15, wherein said mammal is a human.

17. The method of claim 15, wherein said Atopobium species is A. vaginae.

18. The method of claim 15, wherein said Porphyromonas species is P. somerae.

19. The method of claim 15, wherein said Porphyromonas species is a species having 16S rRNA that is greater than 98 percent identical to a 16S rRNA sequence of P. somerae.

20. The method of claim 15, wherein said Peptoniphilus species is P. harei or P. coxii.

21. A method for identifying a female mammal as having endometrial hyperplasia, wherein said method comprises:
(a) detecting the presence of an Atopobium species, a Porphyromonas species, and/or a Peptoniphilus species within the upper reproductive tract of said mammal, but not within the lower reproductive tract of said mammal, and
(b) classifying said mammal as having endometrial hyperplasia.

22. The method of claim 21, wherein said mammal is a human.

23. The method of claim 21, wherein said Atopobium species is A. vaginae.

24. The method of claim 21, wherein said Porphyromonas species is P. somerae.

25. The method of claim 21, wherein said Porphyromonas species is a species having 16S rRNA that is greater than 98 percent identical to a 16S rRNA sequence of P. somerae.

26. The method of claim 21, wherein said Peptoniphilus species is P. harei or P. coxii.

27. A method for treating a female mammal having endometrial cancer, wherein said method comprises:
(a) identifying said mammal as having an Atopobium species and/or a Peptoniphilus species within the lower reproductive tract of said mammal, and
(b) administering an antibiotic to said mammal to reduce the number of Atopobium species bacteria and/or the number of Peptoniphilus species bacteria present within the lower reproductive tract of said mammal.

28. The method of claim 27, wherein said mammal is a human.

29. The method of claim 27, wherein said Atopobium species is A. vaginae.

30. The method of claim 27, wherein said Peptoniphilus species is P. harei or P. coxii.

31. The method of claim 27, further comprising identifying said mammal as having a Porphyromonas species within the lower reproductive tract of said mammal.

32. The method of claim 31, wherein said Porphyromonas species is P. somerae.

33. The method of claim 31, wherein said Porphyromonas species is a species having 16S rRNA that is greater than 98 percent identical to a 16S rRNA sequence of P. somerae.

34. The method of claim 27, wherein said antibiotic is benzylpenicillin, tetracycline, amoxicillin, ampicillin, ticarcillin, piperacillin, cephalothin, cefuroxime, cefotaxime, cefoxitin, imipenem erythromycin, cefamandole, cephaloridine, oleandomycin, metronidazole, spiramycin, or clindamycin.

35. A method for treating a female mammal as having endometrial hyperplasia, wherein said method comprises:
(a) identifying said mammal as having an Atopobium species, a Porphyromonas species, and/or a Peptoniphilus species within the upper reproductive tract of said mammal, but not within the lower reproductive tract of said mammal, and
(b) administering an antibiotic to said mammal to reduce the number of Atopobium species bacteria, the number of Porphyromonas species bacteria, and/or the number of Peptoniphilus species bacteria present within the lower reproductive tract of said mammal.

36. The method of claim 35, wherein said mammal is a human.

37. The method of claim 35, wherein said Atopobium species is A. vaginae.

38. The method of claim 35, wherein said Porphyromonas species is P. somerae.

39. The method of claim 35, wherein said Peptoniphilus species is P. harei or P. coxii.

40. The method of claim 35, wherein said Porphyromonas species is a species having 16S rRNA that is greater than 98 percent identical to a 16S rRNA sequence of P. somerae.

41. The method of claim 35, wherein said antibiotic is benzylpenicillin, tetracycline, amoxicillin, ampicillin, ticarcillin, piperacillin, cephalothin, cefuroxime, cefotaxime, cefoxitin, imipenem erythromycin, cefamandole, cephaloridine, oleandomycin, metronidazole, spiramycin, or clindamycin.

42. The method of claim 35, further comprising administering progestin therapy to said mammal.


SEQUENCE LISTING (7 sequences)

SEQ ID NO: 1 - DNA, 19 bp, artificial
  universal 16S primer
gtcctacgggaggcagcag

SEQ ID NO: 2 - DNA, 29 bp, artificial
  16S 5′ Illumina Adapter
aatgatacggcgaccaccgagatctacac

SEQ ID NO: 3 - DNA, 10 bp, artificial
  16S forward primer pad
tatggtaatt

SEQ ID NO: 4 - DNA, 58 bp, artificial
  16S rDNA forward primer
aatgatacggcgaccaccgagatctacactatggtaattgtcctacgggaggcagcag

SEQ ID NO: 5 - DNA, 18 bp, artificial
  universal 16S primer
ccgtcaattcmtttragt

SEQ ID NO: 6 - DNA, 36 bp, artificial
  reverse complement of 16S 3′ Illumina Adapter
caagcagaagacggcatacgagatgccgcattcgat

SEQ ID NO: 7 - DNA, 66 bp, artificial
  16S rDNA reverse primer
  misc_feature (37)..(48): n is a, c, g, or t
caagcagaagacggcatacgagatgccgcattcgatnnnnnnnnnnnnccgtcaattcmtttragt